(19) United States
(12) Patent Application Publication
(10) Pub. No.: US 2005/0147662 A1
(43) Pub. Date: Jul. 7, 2005

(54) Compounds and compositions for delivering active agents

(75) Inventor: John J. Freeman JR., New Fairfield, CT (US)

Correspondence Address: DARBY & DARBY P.C., P. O. BOX 5257, NEW YORK, NY, 10150-5257, US

(73) Assignee: Emisphere Technologies, Inc., Tarrytown, NY (US)

(21) Appl. No.: 11/018,147

(22) Filed: Dec. 20, 2004

Related U.S. Application Data
(60) Provisional application No. 60/552,337, filed on Mar. 10, 2004.
(60) Provisional application No. 60/530,941, filed on Dec. 19, 2003.

(51) Int. Cl. A61K 38/28; A61K 38/29; A61K 38/26; A61K 31/195; C07C 233/51

(52) U.S. Cl. 424/451; 514/12; 514/567; 562/450; 424/464; 514/3

(57) ABSTRACT

Compounds and compositions for the delivery of active agents are provided. Methods of administration and preparation are provided as well.

This application claims the benefit of U.S. Provisional Application No. 60/552,337, filed Mar. 10, 2004, and U.S. Provisional Application No. 60/530,941, filed Dec. 19, 2003. Both of these applications are hereby incorporated by reference in their entirety.

### FIELD OF THE INVENTION

The present invention relates to compounds for delivering active agents, such as biologically or chemically active agents, to a target. These compounds are well suited for forming non-covalent mixtures with active agents for oral, intracolonic, pulmonary, and other routes of administration to animals. Methods for the preparation and administration of such compositions are also disclosed.

### BACKGROUND OF THE INVENTION

Conventional means for delivering active agents are often severely limited by biological, chemical, and physical barriers. Typically, these barriers are imposed by the environment through which delivery occurs, the environment of the target for delivery, and/or the target itself. Biologically and chemically active agents are particularly vulnerable to such barriers.

In the delivery to animals of biologically active and chemically active pharmacological and therapeutic agents, barriers are imposed by the body. Examples of physical barriers are the skin, lipid bi-layers and various organ membranes that are relatively impermeable to certain active agents but must be traversed before reaching a target, such as the circulatory system. Chemical barriers include, but are not limited to, pH variations in the gastrointestinal (GI) tract and degrading enzymes.

These barriers are of particular significance in the design of oral delivery systems. Oral delivery of many biologically or chemically active agents would be the route of choice for administration to animals if not for biological, chemical, and physical barriers. Among the numerous agents which are not typically amenable to oral administration are biologically or chemically active peptides, such as calcitonin and insulin; polysaccharides, and in particular mucopolysaccharides including, but not limited to, heparin; heparinoids; antibiotics; and other organic substances. These agents may be rapidly rendered ineffective or destroyed in the gastro-intestinal tract by acid hydrolysis, enzymes, and the like. In addition, the size and structure of macromolecular drugs may prohibit absorption.

Earlier methods for orally administering vulnerable pharmacological agents have relied on the co-administration of adjuvants (e.g., resorcinols and non-ionic surfactants such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Liposomes have also been described as drug delivery systems for insulin and heparin. However, broad spectrum use of such drug delivery systems is precluded because: (1) the systems require toxic amounts of adjuvants or inhibitors; (2) suitable low molecular weight cargos, i.e. active agents, are not available; (3) the systems exhibit poor stability and inadequate shelf life; (4) the systems are difficult to manufacture; (5) the systems fail to protect the active agent (cargo); (6) the systems adversely alter the active agent; or (7) the systems fail to allow or promote absorption of the active agent.

Proteinoid microspheres have been used to deliver pharmaceuticals. See, for example, U.S. Pat. Nos. 5,401,516; 5,443,841; and Re. 35,862. In addition, certain modified amino acids have been used to deliver pharmaceuticals. See, for example, U.S. Pat. Nos. 5,629,020; 5,643,957; 5,766,633; 5,776,888; and 5,866,536.

More recently, a polymer has been conjugated to a modified amino acid or a derivative thereof via a linkage group to provide for polymeric delivery agents. The modified polymer may be any polymer, but preferred polymers include, but are not limited to, polyethylene glycol (PEG), and derivatives thereof. See, for example, International Patent Publication No. WO 00/40203.

However, there is still a need for simple, inexpensive delivery systems which are easily prepared and which can deliver a broad range of active agents by various routes.

### SUMMARY OF THE INVENTION

The present invention provides compounds and compositions which facilitate the delivery of active agents. Delivery agent compounds of the present invention include compounds 1 and 2 as shown below and salts thereof:

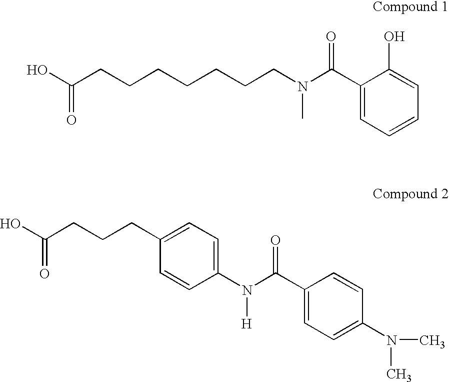

Mixtures of these delivery agent compounds may also be used.

The invention also provides a composition comprising at least one of the delivery agent compounds of the formulas above, and at least one active agent. These compositions deliver active agents to selected biological systems in increased or improved bioavailability of the active agent compared to administration of the active agent without the delivery agent compound.

Also provided are dosage unit forms comprising the compositions. The dosage unit may be in the form of a liquid or a solid, such as a tablet, capsule or particle, including a powder or sachet.

Another embodiment is a method for administering an active agent to an animal, particularly an animal in need of the active agent, by administering a composition comprising at least one of the delivery agent compounds of the formulas above and the active agent to the animal. Preferred routes of administration include the oral and intracolonic routes.

Yet another embodiment is a method of treating a disease or for achieving a desired physiological effect in an animal by administering the composition of the present invention.

Yet another embodiment is a method of preparing a composition of the present invention by mixing at least one delivery agent compound of the formulas above, and at least one active agent.

### DETAILED DESCRIPTION OF THE INVENTION

Delivery Agent Compounds

The terms “alkyl”, “alkenyl”, and “alkynyl” as used herein include linear and branched alkyl, alkenyl, and alkynyl substituents, respectively.

The delivery agent compounds may be in the form of the free base or salts thereof. Suitable salts include, but are not limited to, organic and inorganic salts, for example ammonium, acetate salt, citrate salt, halide (preferably hydrochloride), hydroxide, sodium, sulfate, nitrate, phosphate, alkoxy, perchlorate, tetrafluoroborate, carboxylate, mesylate, fumerate, malonate, succinate, tartrate, acetate, gluconate, and maleate. Preferred salts include, but are not limited to, sodium, citrate and mesylate salts. The salts may also be solvates, including ethanol solvates, and hydrates.

Salts of the delivery agent compounds of the present invention may be prepared by methods known in the art. For example, citrate salts and mesylate salts may be prepared in ethanol, toluene and citric acid.

The delivery agent compound may be purified by recrystallization or by fractionation on one or more solid chromatographic supports, alone or linked in tandem. Suitable recrystallization solvent systems include, but are not limited to, ethanol, water, heptane, ethyl acetate, acetonitrile, acetone, methanol, and tetrahydrofuran (THF) and mixtures thereof. Fractionation may be performed on a suitable chromatographic support such as alumina, using methanol/n-propanol mixtures as the mobile phase; reverse phase chromatography using trifluoroacetic acid/acetonitrile mixtures as the mobile phase; and ion exchange chromatography using water or an appropriate buffer as the mobile phase. When anion exchange chromatography is performed, preferably a 0-500 mM sodium chloride gradient is employed.

The delivery agent may contain a polymer conjugated to it by a linkage group selected from the group consisting of —NHC(O)NH—, —C(O)NH—, —NHC(O), —OOC—, —COO—, —NHC(O)O—, —OC(O)NH—, —CH₂NH —NHCH₂—, —CH₂NHC(O)O—, —OC(O)NHCH₂—, —CH₂NHCOCH₂O—, —OCH₂C(O)NHCH₂—, —NHC(O)CH₂O—, —OCH₂C(O)NH—, —NH—, —O—, and carbon-carbon bond, with the proviso that the polymeric delivery agent is not a polypeptide or polyamino acid. The polymer may be any polymer including, but not limited to, alternating copolymers, block copolymers and random copolymers, which are safe for use in mammals. Preferred polymers include, but are not limited to, polyethylene; polyacrylates; polymethacrylates; poly(oxyethylene); poly(propylene); polypropylene glycol; polyethylene glycol (PEG); and derivatives thereof and combinations thereof. The molecular weight of the polymer typically ranges from about 100 to about 200,000 daltons. The molecular weight of the polymer preferably ranges from about 200 to about 10,000 daltons. In one embodiment, the molecular weight of the polymer ranges from about 200 to about 600 daltons and more preferably ranges from about 300 to about 550 daltons.

Active Agents

Active agents suitable for use in the present invention include biologically active agents and chemically active agents, including, but not limited to, pesticides, pharmacological agents, and therapeutic agents. Suitable active agents include those that are rendered less effective, ineffective or are destroyed in the gastro-intestinal tract by acid hydrolysis, enzymes and the like. Also included as suitable active agents are those macromolecular agents whose physiochemical characteristics, such as, size, structure or charge, prohibit or impede absorption when dosed orally.

For example, biologically or chemically active agents suitable for use in the present invention include, but are not limited to, proteins; polypeptides; peptides; hormones; polysaccharides, and particularly mixtures of muco-polysaccharides; carbohydrates; lipids; small polar organic molecules (i.e. polar organic molecules having a molecular weight of 500 daltons or less); other organic compounds; and particularly compounds which by themselves do not pass (or which pass only a fraction of the administered dose) through the gastro-intestinal mucosa and/or are susceptible to chemical cleavage by acids and enzymes in the gastro-intestinal tract; or any combination thereof.

Further examples include, but are not limited to, the following, including synthetic, natural or recombinant sources thereof: growth hormones, including human growth hormones (hGH), recombinant human growth hormones (rhGH), bovine growth hormones, and porcine growth hormones; growth hormone releasing hormones; growth hormone releasing factor, interferons, including a (e.g., interferon alfacon-1 (available as Infergen® from InterMune, Inc. of Brisbane, Calif.)), β and γ; interleukin-1; interleukin-2; glucagon; insulin, including porcine, bovine, human, and human recombinant, optionally having counter ions including zinc, sodium, calcium and ammonium; insulin-like growth factor, including IGF-1; heparin, including unfractionated heparin, heparinoids, dermatans, chondroitins, low molecular weight heparin, very low molecular weight heparin and ultra low molecular weight heparin; calcitonin, including salmon, eel, porcine and human; erythropoietin; atrial naturetic factor; antigens; monoclonal antibodies; somatostatin; protease inhibitors; adrenocorticotropin, gonadotropin releasing hormone; oxytocin; leutinizing-hormone-releasing-hormone; follicle stimulating hormone; glucocerebrosidase; thrombopoietin; filgrastim; prostaglandins; cyclosporin; vasopressin; cromolyn sodium (sodium or disodium chromoglycate); vancomycin; desferrioxamine (DFO); bisphosphonates, including alendronate, tiludronate, etidronate, clodronate, pamidronate, olpadronate, and incadronate; gallium nitrate; parathyroid hormone (PTH), including its fragments; anti-migraine agents such as BIBN-4096BS and other calcitonin gene-related proteins antagonists; glucagon-like peptide 1 (GLP-1); a dipeptidyl peptidase IV (DPP-4) inhibitor (e.g. LAF237); antimicrobials, including antibiotics, anti-bacterials, anti-virals, and anti-fungal agents; vitamins; pharmaceutically acceptable salts, solvates, active metabolites, prodrugs, racemates, enantiomers, analogs, fragments, mimetics or polyethylene glycol (PEG)-modified derivatives of these compounds; or any combination thereof. Non-limiting examples of antibiotics include gram-positive acting, bacteriocidal, lipopeptidal and cyclic peptidal antibiotics, such as daptomycin and analogs thereof.

Delivery Systems

The composition of the present invention comprises one or more delivery agent compounds of the present invention, and one or more active agents. In one embodiment, one or more of the delivery agent compounds, or salts of these compounds, or poly amino acids or peptides of which these compounds or salts form one or more of the units thereof, may be used as a delivery agent by mixing with the active agent prior to administration to form an administration composition.

The administration compositions may be in the form of a liquid. The solution medium may be water (for example, for salmon calcitonin, parathyroid hormone, and erythropoietin), 25% aqueous propylene glycol (for example, for heparin) and phosphate buffer (for example, for rhGH). Other dosing vehicles include polyethylene glycol. Dosing solutions may be prepared by mixing a solution of the delivery agent compound with a solution of the active agent, just prior to administration. Alternately, a solution of the delivery agent compound (or active agent) may be mixed with the solid form of the active agent (or delivery agent compound). The delivery agent compound and the active agent may also be mixed as dry powders. The delivery agent compound and the active agent can also be admixed during the manufacturing process.

The dosing solutions may optionally contain additives such as phosphate buffer salts, citric acid, glycols, or other dispersing agents. Stabilizing additives may be incorporated into the solution, preferably at a concentration ranging between about 0.1 and 20% (w/v).

The administration compositions may alternately be in the form of a solid, such as a tablet, capsule or particle, such as a powder or sachet. Solid dosage forms may be prepared by mixing the solid form of the compound with the solid form of the active agent. Alternately, a solid may be obtained from a solution of compound and active agent by methods known in the art, such as freeze-drying (lyophilization), precipitation, crystallization and solid dispersion.

The administration compositions of the present invention may also include one or more enzyme inhibitors. Such enzyme inhibitors include, but are not limited to, compounds such as actinonin or epiactinonin and derivatives thereof. Other enzyme inhibitors include, but are not limited to, aprotinin (Trasylol) and Bowman-Birk inhibitor.

The amount of active agent used in an administration composition of the present invention is an amount effective to accomplish the purpose of the particular active agent for the target indication. The amount of active agent in the compositions typically is a pharmacologically, biologically, therapeutically, or chemically effective amount. However, the amount can be less than that amount when the composition is used in a dosage unit form because the dosage unit form may contain a plurality of delivery agent compound/active agent compositions or may contain a divided pharmacologically, biologically, therapeutically, or chemically effective amount. The total effective amount can then be administered in cumulative units containing, in total, an effective amount of the active agent.

The total amount of active agent to be used can be determined by methods known to those skilled in the art. However, because the compositions of the invention may deliver active agents more efficiently than compositions containing the active agent alone, lower amounts of biologically or chemically active agents than those used in prior dosage unit forms or delivery systems can be administered to the subject, while still achieving the same blood levels and/or therapeutic effects.

The presently disclosed delivery agent compounds facilitate the delivery of biologically and chemically active agents, particularly in oral, intranasal, sublingual, intraduodenal, subcutaneous, buccal, intracolonic, rectal, vaginal, mucosal, pulmonary, transdermal, intradermal, parenteral, intravenous, intramuscular and ocular systems, as well as traversing the blood-brain barrier.

Dosage unit forms can also include any one or combination of excipients, diluents, disintegrants, lubricants, plasticizers, colorants, flavorants, taste-masking agents, sugars, sweeteners, salts, and dosing vehicles, including, but not limited to, water, 1,2-propane diol, ethanol, olive oil, or any combination thereof.

The compounds and compositions of the subject invention are useful for administering biologically or chemically active agents to any animals, including but not limited to birds such as chickens; mammals, such as rodents, cows, pigs, dogs, cats, primates, and particularly humans; and insects.

The system is particularly advantageous for delivering chemically or biologically active agents that would otherwise be destroyed or rendered less effective by conditions encountered before the active agent reaches its target zone (i.e. the area in which the active agent of the delivery composition is to be released) and within the body of the animal to which they are administered. Particularly, the compounds and compositions of the present invention are useful for orally administering active agents, especially those that are not ordinarily orally deliverable, or those for which improved delivery is desired.

The compositions comprising the compounds and active agents have utility in the delivery of active agents to selected biological systems and in an increased or improved bioavailability of the active agent compared to administration of the active agent without the delivery agent. Delivery can be improved by delivering more active agent over a period of time, or in delivering the active agent in a particular time period (such as to effect quicker or delayed delivery), or in delivering the active agent at a specific time, or over a period of time (such as sustained delivery).

Another embodiment of the present invention is a method for the treatment or prevention of a disease or for achieving a desired physiological effect, such as those listed in the table below, in an animal by administering the composition of the present invention. Preferably, an effective amount (e.g. a pharmaceutically effective amount) of the composition for the treatment or prevention of the desired disease or for achieving the desired physiological effect is administered. Specific indications for active agents can be found in the Physicians' Desk Reference (58th Ed., 2004, Medical Economics Company, Inc., Montvale, N.J.), and in Fauci, AS, et. al. Harrison's Principles of Internal Medicine (14th Ed., 1998, McGraw-Hill Health Professions Division, New York) both of which are herein incorporated by reference in their entirety. The active agents in the table below include their analogs, fragments, mimetics, and polyethylene glycol-modified derivatives.

| Active Agent | Disease and Physiological Effect |
|---|---|
| Growth hormones (including human recombinant | Growth disorders |
| growth hormone and growth-hormone releasing | |
| factors and its analogs) | |
| Interferons, including α, β and γ. | Viral infection, including chronic cancer, hepatitis, |
| | and multiple sclerosis |
| Innterleukins (e.g. Interleukin-1; interleukin-2) | Viral infection; cancer; cell mediated immunity; and |
| | transplant rejection; |
| Insulin; Insulin-like growth factor IGF-1. | Diabetes |
| Heparin | Treatment and Prevention of Thrombosis, including |
| | (Deep Vein Thrombosis); prevention of blood |
| | coagulation |
| Calcitonin. | Osteoporosis; diseases of the bone; bone pain; |
| | analgesic (including pain associated with |
| | osteoporosis or cancer) |
| Erythropoietin | Anemia; HIV/HIV-therapy Associated Anemia; |
| | Chemotherapeutically-Induced Anemia |
| Atrial naturetic factor | Vasodilation |
| Antigens | Infection |
| CPHPC | Reduction of amyloid deposits and systemic |
| | amyloidoisis often (but not always) in connection |
| | with Alzheimer's disease, Type II diabetes, and other |
| | amyloid-based diseases |
| Monoclonal antibodies | To prevent graft rejection; cancer; used in assays to |
| | detect diseases |
| Somatostatin/octreotide | Bleeding ulcer; erosive gastritis; variceal bleeding; |
| | diarrhea; acromegaly; TSH-secreting pituitary |
| | adenomas; secretory pancreatic tumors; carcinoid |
| | syndrome; reduce proptosis/thyroid-associated |
| | ophthalmopathy; reduce macular |
| | edema/retinopathy |
| Protease inhibitors | HIV Infection/AIDS |
| Adrenocorticotropin | High cholesterol (to lower cholesterol) |
| Gonadotropin releasing hormone | Ovulatory disfunction (to stimulate ovulation) |
| Oxytocin | Labor disfunction (to stimulate contractions) |
| Leutinizing-hormone-releasing-hormone; follicle | Regulate reproductive function |
| stimulating hormone | |
| Glucocerebrosidase | Gaucher disease (to metabolize lipoprotein) |
| Thrombopoietin | Thrombocytopenia |
| Filgrastim (Granulocyte Colony Stimulating | shorten the duration of chemotherapy-induced |
| Factor); GM-CSF, (sargramostim) | neutropenia and thus treat or prevent infection in |
| | chemotherapy patients; Inhibit the growth of or to |
| | kill Mycobacterium Intracellular Avium |
| | Infection (MAC) |
| Prostaglandins | Hypertension |
| Cyclosporin | Transplant rejection |
| Vasopressin | Nocturnal Enuresis; antidiuretic |
| Cromolyn sodium; | Asthma; allergies |
| Vancomycin | Treat or prevent antimicrobial-induced |
| | infections including, but not limitted to methacillin- |
| | resistant Staphalococcus aureus and Staph. |
| | epidermiditis |
| gallium nitrate | Osteoporosis; Paget's disease; Inhibits osteoclasts; |
| | Promotes osteoblastic activity, hypercalcemia, |
| | including cancer related hypercalcemia, urethral |
| | (urinary tract) malignancies; anti-tumors, cancers, |
| | including urethral and bladder cancers; lymphoma; |
| | malignancies (including bladder cancer); leukemia; |
| | management of bone metastases (and associated |
| | pain); muliple myeloma, attenuate immune response, |
| | including allogenic transplant rejections; disrupt iron |
| | metabolism; promote cell migration; wound repair; |
| | to attenuate or treat infectious processes of |
| | mycobacterium species, including but not limited to |
| | mycobacterium tubercolosis, and mycobacterium |
| | avium complex |
| Desferrioxamine (DFO) | Iron overload |
| Parathyroid hormone (PTH), including its | Osteoporosis; |
| fragments. | Diseases of the bone |
| Antimicrobials | Infection including but not limited to gram-positive |
| | bacterial infection |
| Vitamins | Treat and prevent Vitamin deficiencies |
| Bisphosphonates | Osteoporosis; Paget's disease; bone tumors and |
| | metastases (and associated pain); Breast cancer; |
| | including as adjuvant therapy for early stage breast |
| | cancer; management of bone metastases (and |
| | associated pain), including bone metastases associate |
| | with breast cancer, prostate cancer, and lung cancer; |
| | Inhibits osteoclasts; Promotes osteoblastic activity; |
| | treat and/or prevent bone mineral density (bmd) loss; |
| | multiple myeloma; prevention of bone complications |
| | related to malignant osteolysis; fibrous dysplasia; |
| | pediatric osteogenesis imperfecta; hypercalcemia, |
| | urethral (urinary tract) malignancies; reflex |
| | sympathetic dystropy synodrome, acute back pain |
| | after vertebral crush fracture, chronic inflammatory |
| | joint disease, renal bone disease, extrosseous |
| | calcifications, analgesic, vitamin D intoxication, |
| | periarticular ossifications |
| BIBN4096BS - (1-Piperidinecarboxamide. N-[2-[ [ | Anti-migraine; calcitonin gene-related peptide |
| 5-amino-1-[ [4-(4-pyridinyl)-1- | antagonist |
| piperazinyl)carbonyl]pentyl]amino]-1-[ (3,5- | |
| dibromo-4-hydroxyphenyl)methyl]-2-oxoethyl]- | |
| 4(1,4-dihydro-2-oxo-3(2H0-quinazolinyl)-.[R- | |
| (R*,S*)]-) | |
| Glucagon | improving glycemic control (e.g. treating |
| | hypoglycemia and controlling hypoglycemic |
| | reactions), obesity; a diagnostic aid in the radiogical |
| | examination of the stomach, duodenum, small bowel |
| | and colon; Treat acute poisoning With |
| | Cardiovascular Agents including, but not |
| | limited to, calcium channel blockers, beta |
| | blockers |
| GLP-1, Exendin - 3, Exendin - 4 | Diabetes; improving glycemic control (e.g. treating |
| | hypoglycemia and controlling hypoglycemic |
| | reactions), obesity |
| dipeptidyl peptidase IV (DPP-4) inhibitors | Diabetes; improving glycemic control (e.g. treating |
| | hypoglycemia), obesity |
| Peptide YY (PYY) and PYY-like Peptides | Obesity, Diabetes, Eating Disorders, Insulin- |
| | Resistance Syndromes |

For example, one embodiment of the present invention is a method for treating a patient having or susceptible to diabetes by administering insulin and at least one of the delivery agent compounds of the present invention. Other active agents, including those set forth by way of non-limiting example in the above table, can be used in conjunction with the delivery agents of the present invention.

Following administration, the active agent present in the composition or dosage unit form is taken up into the circulation. The bioavailability of the agent can be readily assessed by measuring a known pharmacological activity in blood, e.g. an increase in blood clotting time caused by heparin, or a decrease in circulating calcium levels caused by calcitonin. Alternately, the circulating levels of the active agent itself can be measured directly.

### EXAMPLES

The following examples illustrate the invention without limitation. All parts are given by weight unless otherwise indicated.

Proton nuclear magnetic resonance (¹H NMR) analyses for the compounds listed below were conducted on a 300 MHz Bruker spectrometer using dimethyl sulfoxide (DMSO-d₆) as the solvent unless otherwise indicated.

Liquid chromatograph/mass spectrometry (LC-MS) analyses were performed with an Agilent Technologies, LC/MSD 1100 (single quad) having the following parameters:
Mobile Phase A: 50:950:5 acetonitrile:water:acetic acid (v/v/v)
Mobile Phase B: 950:50:5 acetonitrile:water:acetic acid (v/v/v)
Gradient Elution: 4 minute linear gradient 0-100% B; total time per injection is 11 minutes
Injection volume: 5uL
Column: ZORBAX Rapid Resolution Cartridge, SB-C18, 2.1×30 mm, 3.5 um
Particle size, catalog # 873700-902
Column temp: 40° C.
UV detection at 244 nm
MSD parameters:
   Source: API-ES, positive polarity
   Scan Parameters:
      Mass Range: 125.00-600.00
      Fragmentor: 60 V
      Gain: 1.0 EMV
      Threshold: 150
   Spray Chamber:
      Gas Temp. 350 deg. D
      Drying Gas: 12.0 l/min
      Neb. Pressure; 40 psig
      VCap 4000V positive/negative

### Example 1

### Preparation of Compound 2

Preparation of 4-dimethylamino-benzoyl chloride

To a 1000 mL round bottomed flask was added 4-dimethylamino-benzoic acid (50.0 g, 1.0 eq) and THF (600 mL). A solution of thionyl chloride (44.16 mL, 2.0 eq) in tetrahydrofuran was added and the resulting mixture heated to reflux for 4 hours. The excess thionyl chloride and solvent were removed under reduced pressure to yield 4-dimethylamino-benzoyl chloride as a solid, which was used without further purification in the preparation of compound 560.

Preparation of compound 2: To a 1000 mL round bottomed flask was added chlorotrimethylsilane (15.48 mL, 2 eq) in methylene chloride (250 ml). 4-aminobutyric acid (10.0 g, 1 equivalent) was added and the mixture was heated to reflux for 1.5 hours. The resulting solution was cooled to 0° C. (ice bath) and triethylamine (27.21 mL, 3 equivalents) was added drop-wise. A solution of 4-dimethylamino-benzoyl chloride (11.12 g, 1 eq) in methylene chloride (50 mL) was added drop-wise to the resulting reaction mixture over 0.5 hours. The temperature was maintained at 0° C. (ice bath) during the addition and for 0.5 hour after the addition was complete. The solution was allowed to warm to ambient temperature. Chloroform (25 mL) was added to improve the solubility of the reactants. The reaction was complete (as indicated by TLC) after 16.5 hours. The solvents were removed under reduced pressure. The resulting solid was dissolved in ethyl acetate (500 mL) and 2.5% aqueous sodium bicarbonate (500 mL) was added. The aqueous layer was acidified to pH 6.5 with aqueous sulfuric acid (2 M) and extracted with ethyl acetate (three times 500 mL). After each extraction, the pH of the aqueous layer was adjusted to pH 6.5. The combined ethyl acetate fractions were dried over sodium sulfate. The sodium sulfate was removed by filtration and the solvent removed under reduced pressure. The crude product was recrystallized from methanol/water and dried under reduced pressure to yield compound 560 (4.97 g, approximately 25% overall yield).

### Example 2

### Preparation of Compound 1

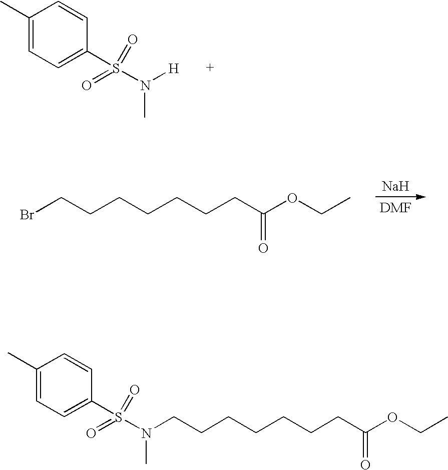

Step 1. 4,N-Dimethylbenzenesulphonamide was reacted with ethyl-8-bromooctanoate in DMF under the influence of sodium hydride to obtain 8-[Methyl-(toluene-4-sulfonyl)-amino]-octanoic acid ethyl ester

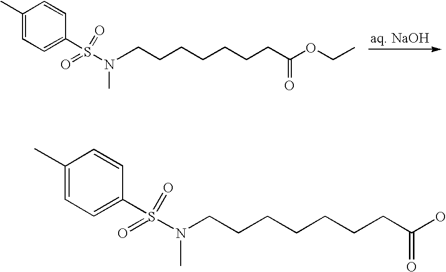

Step 2. The ester of 8-[Methyl-(toluene-4-sulfonyl)-amino]-octanoic acid ethyl ester was hydrolyzed in aqueous sodium hydroxide to obtain 8-[Methyl-(toluene-4-sulfonyl)-amino]-octanoic acid

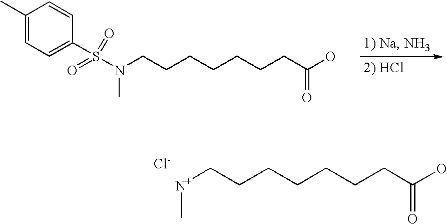

Step 3. The sulphonamide of 8-[Methyl-(toluene-4-sulfonyl)-amino]-octanoic acid was removed under reductive conditions and the resulting amine reacted with hydrogen chloride to obtain (7-Carboxy-heptyl)-methylammonium hydrochloride.

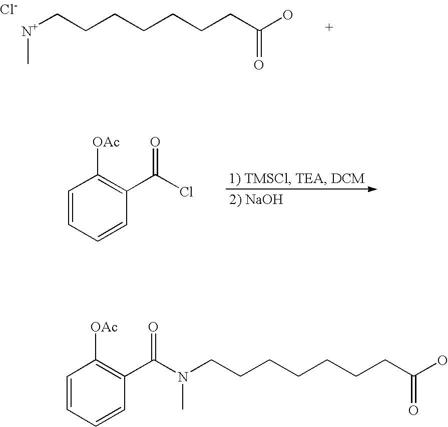

Step 4. The carboxylic acid of (7-Carboxy-heptyl)-methylammonium hydrochloride was protected in-situ with chlorotrimethylsilane. The resulting trimethylsilyl ester was reacted with O-acetylsalicyloyl chloride. The protecting groups were removed with aqueous sodium hydroxide, and after extensive purification, 8-[(2-Hydroxy-benzoyl)-methyl-amino]-octanoic acid, was obtained.

One equivalent each reactants plus 2 equivalents TMSCl plus 2.5 equivalents TEA and MeCl were placed in a 250 ml round bottomed flask fitted with N₂ purge, magnetic stir bar and condenser. TMSCl was added. Heating was begun The reaction mixture was refluxing in an oil bath temperature of 50C after about ½ hour. Heating was stopped after about 2 hours and the reaction mixture placed in an ice/H₂O bath. TEA was added. ASCC was dissolved in 10 ml MeCl2. This was placed in a 60 ml addition funnel atop flask. Dropwise addition was begun. Addition was completed after about ½ hour. The ice bath was removed. Methylene chloride was removed under vacuum. 2N NaOH was added. This was allowed to stir for several hours. Then 2N HCl was added. A yellow oil separated out.

The mixture was extracted 3×100 ml EtOAc. EtOAc was dried with Na2SO4 and concentrated under vacuum. A yellow oil (A) is obtained. 2N NaOH was added to 250 ml round bottom flask containing the yellow oil, the mixture was allowed to stir over the weekend. The mixture was filtered. A tan solid (B) was collected above filter. Below a clear filtrate collected. The filtrate was acidified with 2N HCl. A yellow oil separated. The mixture was extracted 3× with EtOAc. EtOAc is dried with Na2SO4 and concentrated under vacuum. A yellow oil remained (C). The oil was stirred in 40-50C water bath. The aqueous layer was extracted with MeCl2, the MeCl2 was concentrated. A light brown oil was recovered. Oil was taken up in 2N NaOH. A cloudy mixture formed, which was acidified with 2NHCl to pH 5.4, 5.0 and 4.5. At each of these pHs, the aqueous mixture was extracted with 3×50 ml portions EtOAc. The 5.4 and 5.0 fractions were combined, dried with Na2SO4 and concentrated under vacuum. A brown oil was obtained. A number of fractions were found to contain the desired product. These were dissolved in MeCl2 and combined. MeCl2 was removed under vacuum. A brown oil remained, which was taken up in MeOH. Several drops concentrated sulfuric acid were added and the solution allowed to reflux several hours. LC indicated reaction to prepare methyl ester had gone to completion. Heating was stopped. Several mgs sodium bicarbonate were added and MeOH removed under vacuum. The residue was taken up in EtO and extracted first with 2×50 ml portions SAT sodium bicarbonate and then 2×50 ml portions brine. Ether was concentrated and a brown oil remained. The oil was placed on a silica gel column and eluted through column with 70:30 hexane:EtOAc. 100 ml fractions taken. Fractions found to contain desired product by TLC were combined and concentrated. A light colored oil remained. Oil was taken up in about 50 ml 12N NaOH. This was stirred until LC indicated a shift due to hydrolysis of Me ester. The reaction mixture was acidified and a light colored oil separated out. The mixture was extracted with 3×50 ml portions EtOAc, EtOAc was dried with Na2SO4 and concentrated under vacuum. NMR analysis of the oil (A) indicated the oil contained mostly the desired product. SomeEtOAc was present. Upon sitting, the oil slowly solidified. It was placed in refrigeration for about 2 weeks when most had solidified. It was removed from refrigeration and stirred in warm water again. The water was decanted off leaving tan solid (B), which LC indicated contained mostly the desired product with some impurities. An attempt was made to recrystallize from 70:30 Hexane:EtOAc Overnight a tan solid (C) precipitated. This was isolated by filtration and allowed to dry under vacuum overnight. LC of (C) indicated single peak at 4.53. Samples were submitted for analysis, and results were 180C was recrystallized from 70:30 hexane:EtOAc. An oil (180D) separated out, and was taken and isolated. The oil was allowed to stand in a refrigerator. The oil (180D) had begun to crystallize it was allowed to continue to stand in refrigerator. The oil 180D was isolated and some liquid still remained. 180D had a strong acetic acid smell. This was washed several times with H₂O. A tan solid (180E) is isolated. 180E was dried under vacuum overnight. NMR was consistent with desired product. CHN theoretical C=65,31, H=7.82, N=4.76, actual C=65.13, H=8.02, N=4.71. 180E was combined with an earlier fraction and designated as 180F yield 21.95 g.

180F: Yield 2.95 g. Molecular formula C₁₆H₂₃NO₄.

Molecular weight 293 g/mol. Melting point=85-88C. Elemental analysis theoretical: C=65.31, H=7.82, N-4.76; found: C=65.44, H=7.93, N=4.66.

### Example 3

### Preparation of Compound 1

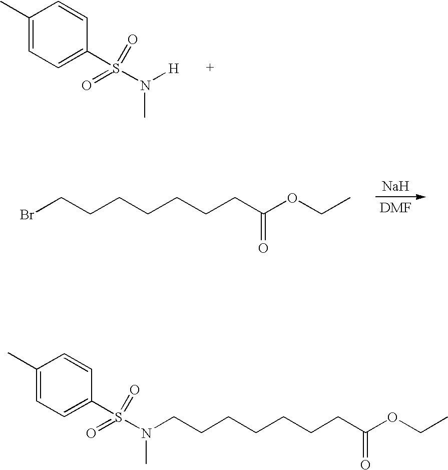

Step 1. 4,N-Dimethylbenzenesulphonamide was reacted with ethyl-8-bromooctanoate in DMF under the influence of sodium hydride to obtain 8-[Methyl-(toluene-4-sulfonyl)-amino]-octanoic acid ethyl ester

Preparation of 8-[Methyl-(toluene-4-sulfonyl)-amino]-octanoic acid ethyl ester: A 500 ml round-bottomed flask equipped with nitrogen purge, magnetic stirbar, and a thermometer was charged with sodium hydride (3.11 g, 0.1297 mol, 1.2 eq) and DMF (30 ml). N-methyl-p-toluenesulphonamide (20.0 g, 0.1081 mol, 1.0 eq) was placed in a 125 ml Erlenmeyer flask and dissolved in DMF (50 ml). The N-methyl-p-toluenesulphonamide solution was added to the sodium hydride mixture dropwise with stirring over the course of approximately 45 min. A water bath was used to maintain the reaction temperature between 23 and 40° C. The resulting reaction mixture was heated to 43° C. for approximately 30 min. In a separate flask, ethyl-8-bromooctanoate (27.14 g, 0.1081 mol, 1.0 eq) was dissolved in DMF (150 ml). The solution of bromoester was added to the reaction mixture dropwise via addition funnel over the course of about 30 min. The reaction was maintained at approximately 58° C. during the addition. The reaction was cooled, and LC indicated completion by one predominant peak corresponding to product. The reaction mixture was poured into ice water (300 ml). The aqueous mixture was extracted with EtOAc (3×200 ml). The combined EtOAc layers were extracted with deionized water (3×200 ml), dried over Na₂SO₄, concentrated under reduced pressure, then placed under high vacuum overnight to yield 36.14 g of crude product. The crude product was chromatographed over silica gel in three portions. Each column was eluted with 80:20 hexane:EtOAc and 125 ml fractions collected. Appropriate product-containing fractions were combined, concentrated under reduced pressure, and further dried under high vacuum. LC and NMR indicated pure product from each of the three columns for a combined yield of 30.21 g of 8-[methyl-(toluene-4-sulfonyl)-amino]-octanoic acid ethyl ester (0.0849 mol, 78.6% yield).

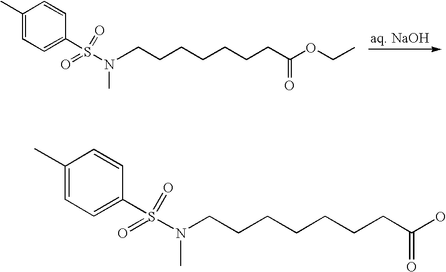

Step 2. The ester of 8-[Methyl-(toluene-4-sulfonyl)-amino]-octanoic acid ethyl ester was hydrolyzed in aqueous sodium hydroxide to obtain 8-[Methyl-(toluene-4-sulfonyl)-amino]-octanoic acid

Preparation of 8-[methyl-(toluene-4-sulfonyl)-amino]-octanoic acid: To a 250 ml round bottomed flask fitted with nitrogen purge and magnetic stir bar was added 8-[methyl-(toluene-4-sulfonyl)-amino]-octanoic acid ethyl ester (10.21 g, 0.0288 mol, 1 eq) and 2N aq. NaOH (57.52 ml, 0.1150 mol, 4.0 eq). The resulting reaction mixture was allowed to stir overnight at ambient temperature. HPLC at this stage still indicated 2 peaks. The reaction mixture was heated to reflux for approximately 6 h, when HPLC indicated reaction was complete, the heat was turned off, and the reaction allowed to cool to ambient temperature overnight. The hazy reaction mixture was acidified with 2N aq. HCl. A white oil separated. The reaction mixture was stirred vigorously in an ice bath and a solid white precipitate formed. The solid was isolated by filtration and dried under vacuum overnight. HPLC indicated a single peak, rt 6.44 min, and NMR was consistent with desired product, 8-[methyl-(toluene-4-sulfonyl)-amino]-octanoic acid: 9.29 g, 0.0284 mol, 98.6% yield.

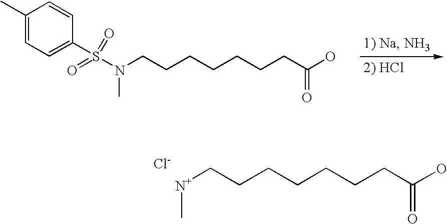

Step 3. The sulphonamide of 8-[Methyl-(toluene-4-sulfonyl)-amino]-octanoic acid was removed under reductive conditions and the resulting amine reacted with hydrogen chloride to obtain (7-Carboxy-heptyl)-methylammonium hydrochloride.

Preparation of (7-carboxy-hepityl)-methylammonium hydrochloride: To a 1000 ml round bottomed flask equipped with a dry ice condenser, nitrogen bubbler, ammonia inlet, and mechanical stirrer was added 8-[methyl-(toluene-4-sulfonyl)-amino]-octanoic acid (9.29 g, 0.0284 mol, 1.0 eq) and THF (20 ml). The mixture was cooled in a dry ice/acetone bath with stirring. Ammonia (ca. 300 ml) was condensed into the flask. Sodium (ca. 3.92 g, 0.1705 mol, 6 eq) was added portion-wise until the blue-green color persisted. Ammonium chloride was added until the reaction mixture appeared white. The dry ice/acetone condenser was removed and the ammonia allowed to boil off overnight. A white solid remained in the flask. Water (10 mls) was added and the mixture was acidified to pH 2-3 by addition of 2N HCl. At this point an oil separated out. The THF was removed under reduced pressure and the aqueous mixture was stirred for about 1 h at ambient temperature. Dichloromethane (50 ml) was added and the solid (product A) was filtered off. The remaining aqueous filtrate was concentrated under reduced pressure to obtain another white solid (product B). NMR analysis indicated that product A is the starting material, 8-[methyl-(toluene-4-sulfonyl)-amino]-octanoic acid, and product B is the desired product, (7-carboxy-heptyl)-methylammonium hydrochloride. The amount of product obtained was greater than 100% of the theoretical mass. Based on the assumption that the product contains sodium chloride and mass balance of recovered starting material, it was assumed the crude product contained 3.72 g of the desired product and it was carried on without further purification.

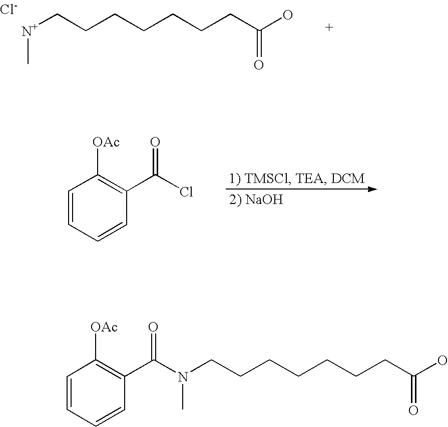

Step 4. The carboxylic acid of (7-Carboxy-heptyl)-methylammonium hydrochloride was protected in-situ with chlorotrimethylsilane. The resulting trimethylsilyl ester was reacted with O-acetylsalicyloyl chloride. The protecting groups were removed with aqueous sodium hydroxide, and after extensive purification, 8-[(2-Hydroxy-benzoyl)-methyl-amino]-octanoic acid, was obtained.

Preparation of 8-[(2-Hydroxy-benzoyl)-methyl-amino]-octanoic acid: To a 100 ml round bottomed flask fitted with argon purge, magnetic stir bar and condenser was added (7-carboxy-heptyl)-methylammonium hydrochloride (3.25 g, 0.0155 mol, 1.0 eq) and dichloromethane (DCM, 50 ml). Chlorotrimethylsilane (3.37 g, 0.0310 mol, 2.0 eq) was added and the resulting mixture was brought to reflux for approximately 2 h. The flask was removed from the heating mantle and placed in an ice water bath. Once the reaction was cooled to 0° C., triethylamine (3.92 g, 0.0388 mol, 2.5 eq) was added and a white vapor formed over the reaction mixture. The reaction was allowed to stir for approximately 10 min at 0° C. In a separate flask, acetylsalicyloyl chloride (ASCC, 3.08 g, 0.0155 mol, 1.0 eq) was dissolved in DCM (20 ml). The ASCC solution was added dropwise to the reaction mixture, the ice bath was removed, and the reaction mixture was allowed to stir and warm to ambient temperature overnight. The DCM was removed under reduced pressure and aqueous 2N NaOH (20 ml) was added to the residue. The aqueous mixture was allowed to stir at ambient temperature for several hours and was then acidified with aqueous 2N HCl. The aqueous mixture became cloudy and a brown oil separated out. The aqueous mixture was extracted with EtOAc (3×100 ml). The combined EtOAc extracts were combined, dried over Na₂SO₄, and concentrated under reduced pressure. The resulting brown oil was further dried under high vacuum to yield a brown solid. HPLC at this point indicated the solid consisted of two components (retention time 2.8 min: salicylic acid, and retention time 4.0 min: desired product). This mixture was stirred in warm water (40-50° C.) to dissolve the salicylic acid. The remaining solid was filtered off. HPLC indicated this is predominantly desired product, crude yield 3.99 g (0.0136 mol, 87.7%). This material was treated in warm water (40-50° C.) and filtered two more times to produce pure 8-[(2-Hydroxy-benzoyl)-methyl-amino]-octanoic acid. (HPLC rt 4.0 min; NMR consistent with desired product; Elemental analysis Theoretical: C=65.31, H=7.82, N=4.76 Found: C=65.32, H=7.72, N=4.73).

All of the above mentioned patents, applications, test methods, and publications are hereby incorporated by reference in their entirety.

Many variations of the present invention will suggest themselves to those skilled in the art in light of the above detailed description. All such obvious variations are within the fully intended scope of the appended claims.

1. A compound selected from: 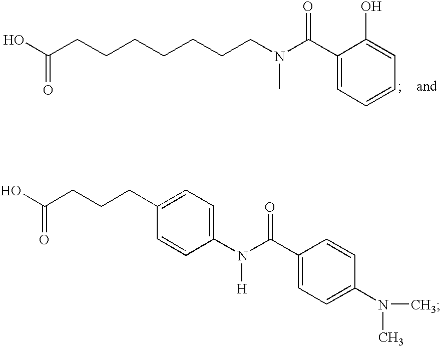
and salts thereof.

2. A composition comprising:
(A) an active agent; and
(B) at least one compound of claim 1.

3. The composition of claim 2, wherein the active agent is selected from the group consisting of a biologically active agent, a chemically active agent, and a combination thereof.

4. The composition of claim 3, wherein the biologically active agent comprises at least one protein, polypeptide, peptide, hormome, polysaccharide, mucopolysaccharide, carbohydrate, or lipid.

5. The composition of claim 3, wherein the biologically active agent is selected from the group consisting of: BIBN-4096BS, growth hormones, human growth hormones recombinant human growth hormones (rhGH), bovine growth hormones, porcine growth hormones, growth hormone releasing hormones, growth hormone releasing factor, CPHPC, interferons, α-interferon, β-interferon, γ-interferon, interleukin-1, interleukin-2, insulin, porcine insulin, bovine insulin, human insulin, human recombinant insulin, insulin-like growth factor (IGF), IGF-1, heparin, unfractionated heparin, heparinoids, dermatans, chondroitins, low molecular weight heparin, very low molecular weight heparin, ultra low molecular weight heparin, calcitonin, salmon calcitonin, eel calcitonin, human calcitonin; gallium nitrate, erythropoietin (EPO), atrial naturetic factor, antigens, monoclonal antibodies, somatostatin, protease inhibitors, adrenocorticotropin, gonadotropin releasing hormone, oxytocin, leutinizing-hormone-releasing-hormone, follicle stimulating hormone, glucocerebrosidase, thrombopoeitin, filgrastim. postaglandins, cyclosporin, vasopressin, cromolyn sodium, sodium chromoglycate, disodium chromoglycate, vancomycin, desferrioxamine (DFO), parathyroid hormone (PTH), fragments of PTH, glucagon, glucagon-like peptide 1 (GLP-1), a dipeptidyl peptidase IV (DPP-4) inhibitor, antimicrobials, anti-fungal agents, vitamins; and pharmacologically acceptable salts, solvates, active metabolites, prodrugs, racemates, enantiomers analogs, fragments, mimetics and polyethylene glycol (PEG)-modified derivatives of these compounds; and any combination thereof.

6. The composition of claim 3, wherein the biologically active agent comprises insulin, BIBN-4096BS, calcitonin, parathyroid hormone, erythropoietin, glucagon, CPHPC, growth hormones or combinations thereof.

7. The composition of claim 6, wherein the biologically active agent comprises glucagon-like peptide 1.

8. The composition of claim 7, further comprising a dipeptidyl peptidase IV (DPP-4) inhibitor.

9. A dosage unit form comprising:
(A) the composition of claim 2; and
(B) (a) an excipient,
(b) a diluent,
(c) a disintegrant,
(d) a lubricant,
(e) a plasticizer,
(f) a colorant,
(g) a dosing vehicle, or
(h) any combination thereof.

10. The dosage unit form of claim 9, wherein the active agent is selected from the group consisting of a biologically active agent, a chemically active agent, and a combination thereof.

11. The dosage unit form of claim 10, wherein the biologically active agent comprises at least one protein, polypeptide, peptide, hormone, polysaccharide, mucopolysaccharide, carbohydrate, or lipid.

12. The dosage unit form of claim 10, wherein the biologically active agent is selected from the group consisting of: BIBN-4096BS, growth hormones, human growth hormones (hGH), recombinant human growth hormones (rhGH), bovine growth hormones, porcine growth hormones, growth hormone releasing hormones, growth hormone releasing factor, interferons, α-interferon, β-interferon, γ-interferon, interleukin-1, interleukin-2, insulin, porcine insulin, bovine insulin, human insulin, human recombinant insulin, insulin-like growth factor, insulin-like growth factor-1, heparin, unfractionated heparin, heparinoids, dermatans, chondroitins, low molecular weight heparin, very low molecular weight heparin, ultra low molecular weight heparin, calcitonin, salmon calcitonin, eel calcitonin, human calcitonin; gallium nitrate; erythropoietin, atrial naturetic factor, antigens, monoclonal antibodies, somatostatin, protease inhibitors, adrenocorticotropin, gonadotropin releasing hormone, oxytocin, leutinizing-hormone-releasing-hormone, follicle stimulating hormone, glucocerebrosidase, thrombopoeitin, filgrastim. postaglandins, cyclosporin, vasopressin, cromolyn sodium, sodium chromoglycate, disodium chromoglycate, vancomycin, desferrioxamine, parathyroid hormone, fragments of PTH, glucagon-like peptide 1 (GLP-1), a dipeptidyl peptidase IV (DPP-4) inhibitor, antimicrobials, anti-fungal agents, vitamins; and pharmacologically acceptable salts, solvates, active metabolites, prodrugs, racemates, enantiomers analogs fragments, mimetics and polyethylene glycol-modified derivatives of these compounds; and any combination thereof.

13. The dosage unit form of claim 10, wherein the biologically active agent comprises insulin, BIBN-4096BS, calcitonin, parathyroid hormone, erythropoietin, glucagon, CPHPC, human growth hormones or combinations thereof.

14. The dosage unit form of claim 10, wherein the active agent comprises insulin.

15. The dosage unit form of claim 10, wherein the active agent comprises glucagon-like peptide 1.

16. The dosage unit form of claim 15, further comprising a dipeptidyl peptidase IV (DPP-4) inhibitor.

17. The dosage unit form of claim 9, wherein the dosage unit form comprises a dosing vehicle comprising a tablet, a capsule, a powder, or a liquid.

18. The dosage unit form of claim 9, wherein the dosing vehicle is a liquid selected from the group consisting of water, 1,2-propane diol, ethanol, and any combination thereof.

19. A method for administering a biologically-active agent to an animal in need of the agent, the method comprising administering orally to the animal the composition of claim 2.

20. A method for preparing a composition comprising mixing:
(A) at least one active agent;
(B) at least one compound of claim 1; and
(C) optionally, a dosing vehicle.
